(19) United States
(12) Patent Application Publication
(10) Pub. No.: US 2018/0200544 A1
(43) Pub. Date: Jul. 19, 2018

(54) BREATHING GLASSES FOR PURIFYING AIR

(71) Applicant: Qianyi LIU, Guangzhou City (CN)

(72) Inventor: Qianyi LIU, Guangzhou City (CN)

(21) Appl. No.: 15/573,277

(22) PCT Filed: May 17, 2016
(86) PCT No.: PCT/CN2016/000263

§ 371 Date: Nov. 10, 2017

(30) Foreign Application Priority Data
May 20, 2015 (CN) 201510256866.3

(51) Int. Cl. A62B 7/10; A61F 9/02; B01D 53/02; B01D 39/04; B01D 46/00; B01D 46/54; A62B 9/00; A62B 9/06; A62B 23/02; G02C 11/00; G02C 5/14

(52) U.S. Cl. CPC A62B 7/10; A61F 9/029; B01D 53/02; B01D 39/04; B01D 46/0038; B01D 46/0019; B01D 2267/40; A62B 9/00; A62B 9/06; A62B 23/02; G02C 11/00; G02C 5/143; B01D 2253/102; B01D 46/54

(57) ABSTRACT

Breathing glasses for purifying air include left and right air filters, an air transmission pipe at the left and right sides thereof, an air curtain ejection and air transmission device and an eye protection cover. The left and the right air filters include a filter casing, a composite filter layer, a built-in air inductor, an intelligent induction chip, a dual-channel variable-frequency turbofan and a miniature motor, a battery and a power control switch, and a lower air transmission transformation connection port. The air curtain ejection and air transmission device ejects air through an upper air curtain outlet groove, a lower air curtain air outlet groove and two breathing air transmission holes to form an air curtain isolation layer to block surrounding unclean air, clean air is ejected from the two breathing air transmission holes. A long-time wearing mode is provided when a portable external air filter is included.

### RELATED APPLICATIONS

The present application is a National Phase of International Application Number PCT/CN2016/000263, May 17, 2016, and claims the priority of China Application No. 201510256866.3, filed May 20, 2015.

### TECHNICAL FIELD OF THE INVENTION

The present invention relates to the field of an air purification apparatus, and more particularly to breathing glasses for purifying air.

### TECHNICAL BACKGROUND OF THE INVENTION

At present, due to vehicle exhaust, factory emissions, storms and other reasons, many cities' air is filled with dust, smoke, plant pollen, spores, bacteria, fine particles, etc., the polluted air not only affects people's travel and fitness but also causes a great threat on people's physical health. In order to avoid the inhalation of the polluted air, some people use a mask, etc. to filter the air, the commonly used various structures of the masks are used to cover the nose and mouth and most of a face with a large area of materials, therefore, the user's facial expressions cannot be shown, affecting the communication between people, furthermore, these structures of the masks' wearing comfort is not ideal, there will be suffocating feeling and breathe obstruction, the exhaled emissions and pollutants will form new pollution in the inner layer of the mask.

### SUMMARY OF THE PRESENT INVENTION

In view of the above-mentioned problems and deficiencies, the present invention aims at providing breathing glasses which do not affect facial expressions and facilitate the people to communicate and be capable of breathing purified air.

The technical solution of the present invention is realized as follows: breathing glasses for purifying air comprises an ear lug type left air filter and an ear lug type right air filter, a left side air transmission pipe and a right side air transmission pipe which are connected with the left air filter and the right air filter respectively, an air curtain ejection and air transmission device connected with the lower ends of the left side air transmission pipe and the right side air transmission pipe, and an eye protection cover, wherein the left air filter and the right air filter each comprise a filter casing, a composite filter layer disposed within the filter casing, a built-in air inductor, an intelligent induction chip, a dual-channel variable-frequency turbofan, a cavity-shaped based made of a flexible material, a miniature motor and a battery, the filter casing is provided thereon with an air inlet, a heat dissipation exhaust port, an inclined exhaust trench, as well as a power control switch and a lower air transmission transformation connection port which are integrally designed with the flexible material.

Further, in order to block the polluted air from affecting the eyes, and also to prevent the eyes from being ejected by the filtered air, the eye protection cover comprises an integral eye protection lens made of transparent material, such as an integral PC eye protection lens, and a bracket provided on the both sides of the integral PC eye protection lens, the integral PC eye protection lens forms a wearing support frame through the brackets at the two sides thereof that are connected with the left side air transmission pipe and the right side air transmission pipe, respectively.

Further, an air curtain ejection and air transmission device with a three-dimensional multi-curved surface is adopted, the air curtain ejection and air transmission device comprises an upper air curtain air outlet groove, a lower air curtain air outlet groove, two breathing air transmission holes and an exhalation pressure or temperature difference induction trigger element, the air exhalation pressure or temperature difference induction trigger element is connected with the intelligent induction chip in a vortex housing of the left air filter and the right air filter, the intelligent induction chip detects the respiration rate of a user and air quality, and controls the peak value of the air volume of a variable-frequency nozzle, the exhalation pressure or temperature difference induction trigger element triggers once every time when the user exhales, controls the dual-channel variable-frequency turbofan in the left air filter and the right air filter to delay for a time setting of 0.8-1.2 seconds, then controls the turbofan to rotate at a high speed for a specific time and transfers air to the air curtain ejection and the air transmission device via the left side air transmission pipe and the right side air transmission pipe, the filtered clean air is ejected from the upper air curtain air outlet groove, the lower air curtain air outlet groove and the two breathing air transmission holes of the air curtain ejection and air transmission device, the air ejected from the upper air curtain air outlet groove and the lower air curtain air outlet groove forms an air curtain insulation layer which blocks the surrounding unclean air, a small amount of clean air, which is continuously ejected from the two breathing air transmission holes, causes the nostril portions of the user to form a miniature positive pressure zone to better isolate the surrounding polluted air; a large amount of clean air ejected from the two breathing holes synchronously at a suction rhythm by a variable frequency is absorbed by the user.

In order that the user's inspiratory rhythm is synchronized, the two-channel variable frequency turbofan in the left air filter and the right air filter is controlled to delay for one second, then be rotated at a high speed and transfer the air to the air curtain ejection and air transmission device for 2-3 seconds through the left side air transmission pipe and the right side air transmission pipe.

Further, the air curtain ejection device comprises a rigid pipe body, both ends of the rigid pipe body are communicated with the left side air transmission pipe and the right side air transmission pipe, respectively, the outer side of the rigid pipe body is provided with a rigid caulking groove, the cross section of the inner portion of the rigid caulking groove is a circular arc shape, the neck of the rigid caulking groove is reduced, an opening of the outer portion of the rigid caulking groove is enlarged, the value of the angle range of the opening of the outer side of the rigid caulking groove is between 140°-170°. the rear portion of the rigid caulking groove is provided with an air transmission channel communicated with a pipe cavity of the rigid pipe body, a flexible air curtain guide plate made of the flexible material (for example, silica) is embedded in the rigid caulking groove, a gap is remained between the outer wall of the flexible air curtain guide plate and the inner wall of the rigid caulking groove to form an air curtain channel, the flexible air curtain guide plate is provided with a multi-point anchor adapted to the rigid caulking groove, the flexible air curtain guide plate is blocked and secured in the rigid caulking groove via the multi-point anchor, the flexible air curtain guide plate and the gap at the opening of the rigid caulking groove form the upper air curtain air outlet groove and the lower air curtain air outlet groove, the two breathing air transmission holes are provided at the upper portion of the rigid pipe body corresponding to nostrils, the exhalation pressure or temperature difference induction trigger element is provided between the two breathing air transmission holes.

Further, the integral PC eye protection lens is built in with a visual air monitoring display wafer and an outdoor exercise display wafer, the outer walls of the left side air transmission pipe and the right side air transmission pipe are covered with a C-shaped plastic plate buckle, the integral PC eye protection lens is embedded and blocked into a slot between the C-shaped plastic plate buckle and the walls of the left side air transmission pipe and the right side air transmission pipe through the brackets at the two sides thereof respectively to be integrally connected with the left side air transmission pipe and the right side air transmission pipe. In order to make full use of solar energy, the left and the right portions of the lens may also be affixed with a solar photovoltaic film as an additional supplement power supply.

Further, the outer surface of the flexible air curtain guide plate is provided as an arc surface matched with a nose shape, the outer arc surface of the flexible air curtain guide plate coincides with the missing arc surface at the opening of the rigid caulking groove of the rigid pipe body, forming that an air outlet groove in different parts presents a three-dimensional multi-angle-change multi-curved-surface air curtain layer matched with the nose shape and part of a cheek contour along a notch direction. The composite filter layer comprises an outermost mesh coarse filter layer, an intermediate layer ultrafine filter layer and a bottom activated carbon odor filter layer. the left side air transmission pipe and the right side air transmission pipe comprise an air transmission pipe bracket made of a hard plastic or a composite material, the dual-channel variable-frequency turbofan is made of the flexible material, for example, the silica, etc., the transformation connection port at the two sides is made of the flexible material, for example, the silica, etc. an air transmission pipe bracket made of the hard plastic or the composite material functions as a frame integrally fixing the filter casing; the filter casing made of the silica and other flexible materials may improve structural airtight, the flexible slot design may eliminate the installation of various screws to prevent vibration and noise transmission, and have the comfort of contact with the body; the dual-channel variable frequency turbofan made of the silica and other flexible materials reduces working noise.

Further, the filter casing comprises a bottom vortex housing and a metal mussel-shaped gill-like air inlet housing which are made of the flexible material such as silica, and the like, the air inlet housing in a mussel-shaped gill-like structure is provided with an exhaust trench inclining backwards, a large number of hidden tiny air inlet holes is distributed in the air inlet trench, making the appearance more beautiful in a whole, an inner side silica inclining exhaust trench contacted with a head may reduce the perspiration of the skin at a contact surface, the dual-channel variable-frequency turbofan is provided with dual main channels and a central auxiliary channel, the main channel of the dual-channel variable-frequency turbofan is always kept to be rotated at a low speed and transfer the air without being triggered when in an operating state; the miniature motor is mounted on a cavity-shaped embedded type base made of the flexible material, for example, the silica, and the like, a small amount of the transferred air in the center auxiliary channel of the dual-channel variable-frequency turbofan is used to cool the miniature motor, the cavity-shaped embedded type base made of the flexible material reduces the vibration and noise of the miniature motor, the cavity-shaped built-in intelligent induction chip carries out synchronization cooling, the outer portion of the cavity-shaped embedded type base made of the flexible material (for example, the silica, and the like) is provided with a heat dissipation vent distributed in the inclined trench.

Furthermore, for the people who work in a working environment with a large amount of dust at the site, for a variety of different environment-intensive people who are prone to airborne diseases, for the people who work in an unclean air environment for a long time, and for seasonal pollen allergy patients and patients with a lung disease, the present invention has a better protective effect. The present invention further comprises a portable external air filter, the portable external air filter device comprises an intelligent purification integral box, a rechargeable battery arranged in the intelligent purification integral box, a chip and integral circuit module, a centrifugal turbofan vortex housing, and a variable-frequency centrifugal turbofan, the inner portion of the intelligent purification integral box is provided with a coconut-shell activated carbon filter layer, the outer side of the coconut-shell activated carbon filter layer is wrapped with a composite filter layer in a roll-like structure, the side surface of the intelligent purification integral box is provided with a touch control key, the touch control key is provided therein with a wireless data induction signal source, the lower part of the intelligent purification integral box is provided with a water splashing prevention air inlet channel, the water splashing prevention air inlet channel is provided therein with a foam cotton filter layer, the intelligent purification integral box is provided therein with a containable stretchable annular coil pipe, the intelligent purification integral box is provided with an internal pipe connection port, the intelligent purification integral box is provided with a strap on the back side thereof, which is tied to an arm when used.

The lower portions of the left air filter and the right air filter are provided with the transformation connection port, the external connection end of the built-in annular coil pipe extends out from the upper portion of the intelligent purification integral box, when in connection, the external connection end of the annular coil pipe is connected with the transformation connection port provided at the lower portion of the left air filter or the right air filter via a transformation connection head after stretching, and converts a working mode of 8-12 hour air transmission of the externally connected intelligent purification integral box; when not in connection, the annular coil pipe is accommodated in the intelligent purification integral box.

Further, in order that the portable external air filter is enabled to be wirelessly controlled by a mobile phone or a remote controller, the touch control key of the portable external air filter is provided therein with a wirelessly transmitting device, a wireless signal receiving device and a wireless intelligent interactive data processing chip platform.

For the invention, as the air curtain ejection and air transmission device are small in volume, and placed between a mouth and the nose, and the filtered air is transported through the left side air pipe and the right side air pipe, the invention has the advantages of a small area of the covered face, no impact on face expressions, fully weakening the impact on facial appearance relative to the traditional mask, and no affecting people to communicate. the air ejected from the upper air curtain air outlet groove and the lower air curtain air outlet groove forms an air curtain insulation layer which blocks the surrounding unclean air, the air ejected from the two breathing air transmission holes is sucked by the user, the human respiratory rate is synchronized, which improves the comfort while also reducing the consumption of electricity to protect the health of the person. The present invention is used as a short time wearing mode, that is, is used for 1-2 hours of working time, for example, if the glasses are used in urban public places and traffic, the glasses may reduce the body's inhalation of polluted air. For the city's outdoor sports, the glasses have a good protective effect. For a long-time wearing mode, that is, when the glasses are used for 8-12 hours of the working time, the portable external air filter, an ultra-long time power supply and a more powerful multi-layer air filtration system are added, which has a good protective effect for the seasonal pollen allergy patients, the patients with the lung disease, a traffic policeman on duty and the people who work at the site and in the working environment with a large number of dust; the glasses have a protective effect on the prevention of airborne infectious diseases.

The invention is further described with reference to the following accompanying drawings.

### BRIEF DESCRIPTION OF THE DRAWINGS

FIG. 1 is a structural schematic view of a short time wearing mode of the present invention;

FIG. 2 is a structural schematic view of a plan view (partially sectional view) of the present invention;

FIG. 3 is a structural schematic diagram of a principle of a variable-frequency turbofan air transmission working path in a short-time wear mode of the present invention (arrow direction is the variable-frequency turbofan air transmission working path)

FIG. 4 is a structural schematic view of a plan view of the present invention;

FIG. 5 is a structural schematic view of one perspective view of the present invention;

FIG. 6 is a structural schematic view of a front view of the present invention;

FIG. 7 is a structural schematic view (partial enlargement) of an air curtain ejection and air transmission device of the present invention;

FIG. 8 is a structural schematic view showing the use effect of the present invention;

FIG. 9 is a front schematic view of a short time wearing mode within a 1-2 hour working time of the present invention;

FIG. 10 is a schematic diagram of a short time wearing mode within a 1-2 hour working time of the present invention;

FIG. 11 is a structural schematic view of a portable external air filter according to the present invention;

FIG. 12 is a structural schematic view of one view of a portable external air filter according to the present invention;

FIG. 13 is a structural schematic cross-sectional view of a portable external air filter according to the present invention;

FIG. 14 is a structural schematic view of a long time wearing mode of the present invention;

FIG. 15 is a schematic view of a long-time wearing mode within 8-12 hour working time of the present invention (a transformation connection port is locally enlarged).

### EMBODIMENTS OF THE PRESENT INVENTION

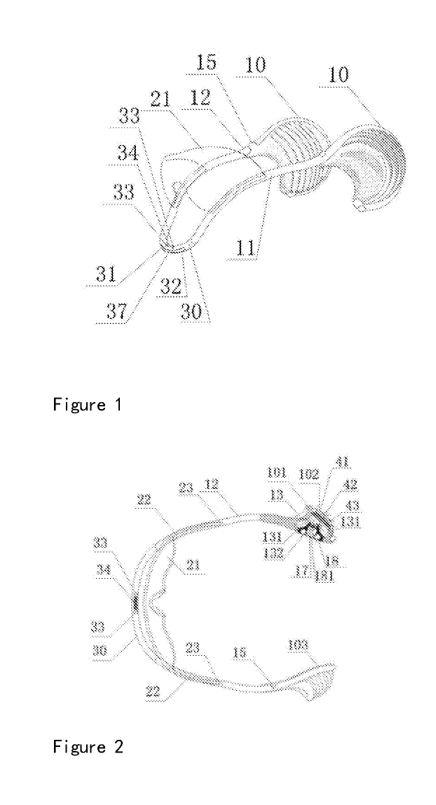

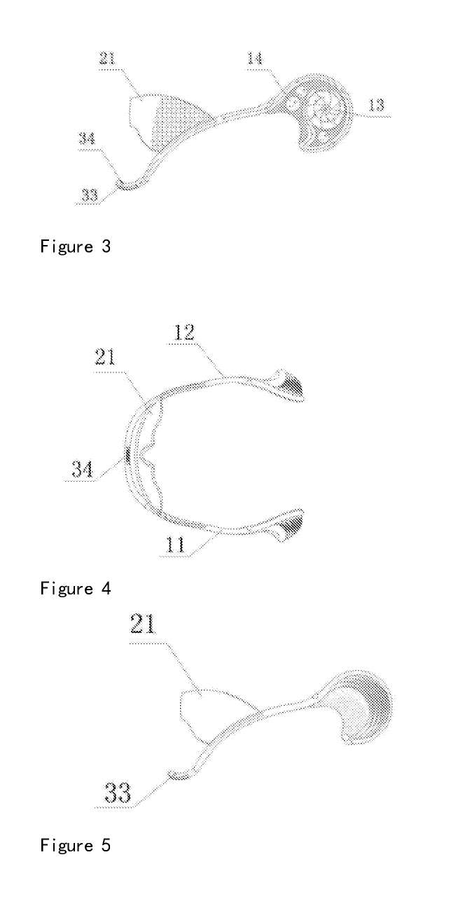

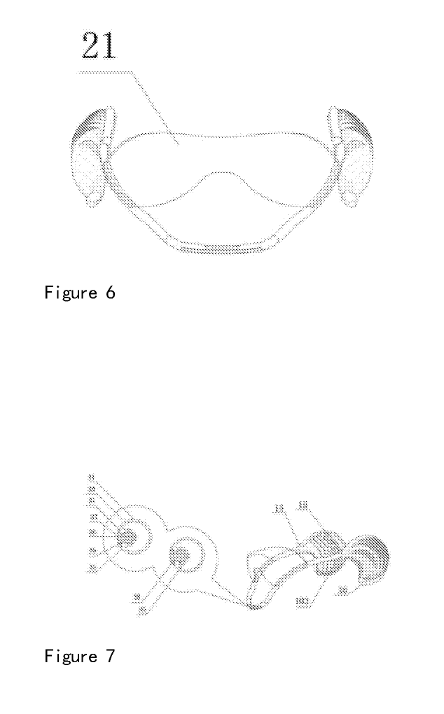

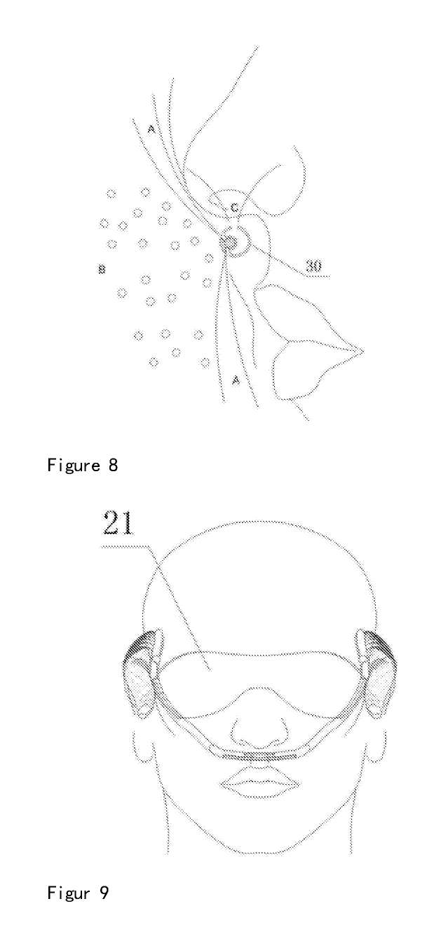

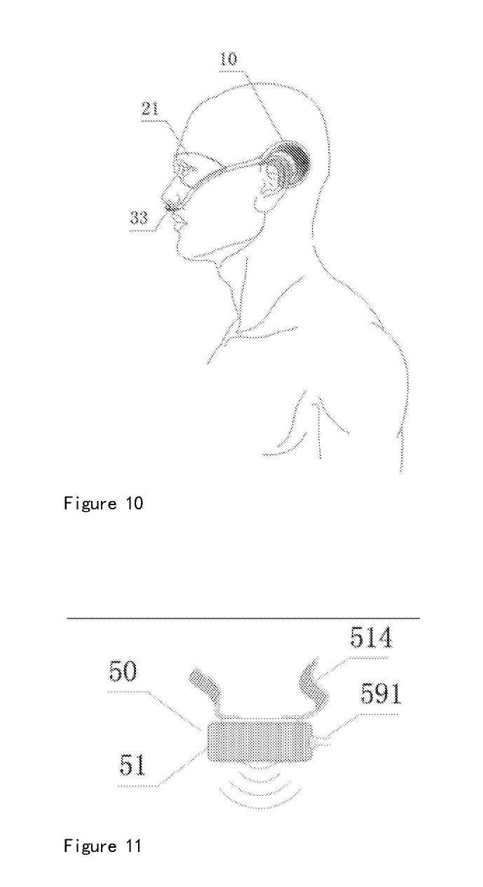

As shown in FIGS. 1-10, The present invention is breathing glasses for purifying air comprises an ear lug type left air filter and an ear lug type right air filter, a left side air transmission pipe 11 and a right side air transmission pipe 12 which are connected with the left air filter and the right air filter respectively, an air curtain spray and air transmission device connected with the lower ends of the left side air transmission pipe 11 and the right side air transmission pipe 12, and an eye protection cover, wherein the left air filter and the right air filter each comprise a filter casing, a composite filter layer disposed within the filter casing, a built-in air inductor, an intelligent induction chip, a dual-channel variable-frequency turbofan 13, a miniature motor 17, a drive mechanism and a battery 14, a battery 14 may be a disposable battery or a rechargeable battery, for example, the battery may be a lithium power supply, a dry battery and a button battery which may be stored with about one hour of electric quantity, etc. the filter casing is provided thereon with an air inlet, an integral transformation connection air transmission port and an integral power control switch 15; the eye protection cover comprises an integral PC eye protection lens 21 and a bracket 22 provided on the both sides of the integral PC eye protection lens 21, the integral PC eye protection lens 21 forms a wearing support frame through the brackets 22 at the two sides thereof that are connected with the left side air transmission pipe 11 and the right side air transmission pipe 12, respectively. the air curtain ejection and air transmission device comprises an upper air curtain air outlet groove 31, a lower air curtain air outlet groove 32, two breathing air transmission holes 33 and an exhalation pressure or temperature difference induction trigger element 34, the air exhalation pressure or temperature difference induction trigger element 34 is connected with the intelligent induction chip in the left air filter and the right air filter, the intelligent induction chip detects the respiration rate of a user and air quality, and controls the peak value of the air volume of a variable-frequency nozzle, the exhalation pressure or temperature difference induction trigger element triggers once every time when the user exhales, controls the dual-channel variable-frequency turbofan 13 in the left air filter and the right air filter to delay for a time setting of 0.8-1.2 seconds 11, then controls the turbofan to rotate at a high speed for a specific time and transfers air to the air curtain ejection and the air transmission device via the left side air transmission pipe 11 and the right side air transmission pipe 12, the filtered clean air is ejected from the upper air curtain air outlet groove 31, the lower air curtain air outlet groove 32 and the two breathing air transmission holes 33 of the air curtain ejection and air transmission device, the air ejected from the upper air curtain air outlet groove 31 and the lower air curtain air outlet groove 32 forms a 140°-170° three-dimensional air curtain insulation layer A which blocks the surrounding unclean air B, a small amount of clean air C, which is continuously ejected from the two breathing air transmission holes 33, causes the nostril portions of the user to form a miniature positive pressure zone to better isolate the surrounding polluted air. a large amount of clean air C ejected from the two breathing holes synchronously at a suction rhythm by a variable frequency is sucked by the user.

According to the preferred embodiment of the present invention, the two-channel variable frequency turbofan in the left air filter and the right air filter is controlled to delay for one second, then be rotated at a high speed and transfer the air to the air curtain ejection and air transmission device for 2-3 seconds through the left side air transmission pipe 11 and the right side air transmission pipe 12, which is synchronized with the user's breathing rhythm. The exhalation pressure or temperature difference induction trigger element 34 inducts a breathing frequency and drives the dual-channel variable-frequency turbofan 13 to dynamically supply the amount of the clean air. When in specific use, still according to the situation of different users and the respiratory rate, a variable frequency mode is adjusted to supply a different amount of the clean air.

As shown in FIGS. 1-5, 7, the air curtain ejection device of the present invention comprises a rigid tube body 30, the both ends of the rigid pipe body 30 are communicated with the left side air transmission pipe 11 and the right side air transmission pipe 12, respectively, the both ends of the rigid body 30 are detachably connected to the left side air transmission pipe 11 and the right side air transmission pipe 12 to periodically clean the air curtain ejecting device, the outer side of the rigid pipe body 30 is provided with a rigid caulking groove 35, the cross section of the inner portion of the rigid caulking groove 35 is a circular arc shape, the neck of the rigid caulking groove 35 is reduced, an opening of the outer portion of the rigid caulking groove 35 is enlarged, the angle range of the opening of the outer portion of the caulking groove 35 of the present invention varies between 140° and 170°. According to the preferred embodiment of the present invention, the angle range of the opening of the outer portion of the caulking groove 35 is controlled to vary between 150° and 165°. the rear portion of the rigid caulking groove 35 is provided with an air transmission channel 36 communicated with a pipe cavity of the rigid pipe body 30, a flexible air curtain guide plate 37 made of a flexible material such as silica and the like is embedded in the rigid caulking groove 35, an air curtain channel 39 formed by a gap is remained between the outer wall of the flexible air curtain guide plate 37 and the inner wall of the rigid caulking groove 35, the flexible air curtain guide plate 37 is provided with a multi-point anchor 38 adapted to the rigid caulking groove 35, the flexible air curtain guide plate 37 is blocked and secured in the rigid caulking groove 35 via the multi-point anchor 38, the flexible air curtain guide plate 37 and the gap at the opening of the rigid caulking groove 35 form the upper air curtain air outlet groove 31 and the lower air curtain air outlet groove 32, the two breathing air transmission holes 33 are provided at the upper portion of the rigid pipe body 30 corresponding to nostrils, the exhalation pressure or temperature difference induction trigger element 34 is provided between the two breathing air transmission holes 33.

As shown in FIG. 2, the integral PC eye protection lens 21 of the present invention is provided therein with a visual air monitoring display wafer and an outdoor exercise display wafer, the outer walls of the left side air transmission pipe 11 and the right side air transmission pipe 12 are covered with a C-shaped plastic plate buckle 23, the integral PC eye protection lens 21 is embedded and blocked into a slot between the C-shaped plastic plate buckle 23 and the walls of the left side air transmission pipe 11 and the right side air transmission pipe 12 through the brackets 22 at the two sides thereof respectively to be integrally connected with the left side air transmission pipe 11 and the right side air transmission pipe 12. The lower portion of the PC eye protection lens 21 is provided with an outline thereof coinciding with the bridge of the nose. In order to make full use of solar energy, the left and the right portions of the lens may also be affixed with a solar photovoltaic film as an additional supplement power supply.

As shown in FIGS. 2, 7: the outer surface of the flexible air curtain guide plate 37 of the present invention is provided as an arc surface matched with a nose shape, the outer arc surface of the flexible air curtain guide plate 37 coincides with the missing arc surface at the opening of the rigid caulking groove 35 of the rigid pipe body 30, forming that an air outlet groove in different parts presents a three-dimensional multi-angle-change multi-curved-surface air curtain layer matched with the nose shape and part of a cheek contour along a notch direction. The composite filter layer comprises an outermost mesh coarse filter layer 41, an intermediate layer ultrafine filter layer 42 and a bottom active carbon odor filter layer 43. The left air transmission pipe 11 and the right air transmission pipe 12 comprise an air transmission pipe bracket made of a hard plastic or a composite material, the dual-channel variable-frequency turbofan 13 is made of the flexible material, the transformation connection ports 16 at the two sides thereof are made of the flexible material. the filter casing comprises a flexible material bottom vortex housing 101 and a metal mussel-shaped gill-like inlet housing 102, the air inlet housing 102 in a mussel-shaped gill-like structure is provided with an air inlet trench reclining backwards, a large number of air inlet holes is hidden in the air inlet trench, making the appearance more beautiful in a whole, and no affecting an auricle during air inlet. an inner side inclining flexible exhaust trench 103 contacted with a head may reduce the perspiration of the skin at a contact surface, the dual-channel variable-frequency turbofan 13 is provided with dual main channels 131 and a central auxiliary channel 132, the dual main channels 131 of the dual-channel variable-frequency turbofan 13 are always kept to be rotated at a low speed and transfer the air without being triggered when in an operating state; the miniature motor 17 is mounted on a cavity-shaped embedded type base 18 made of the flexible material, a small amount of the transferred air in the center auxiliary channel 132 of the dual-channel variable-frequency turbofan 13 is used to cool the miniature motor 17, the cavity-shaped embedded type base 18 made of the flexible material reduces the vibration and noise of the miniature motor 17, the built-in intelligent induction chip dissipates heat synchronously, the outer portion of the cavity-shaped embedded type base 18 made of the flexible material is provided with a heat dissipation vent 181 distributed in the inclined trench. The above flexible material is for example silica, and the like.

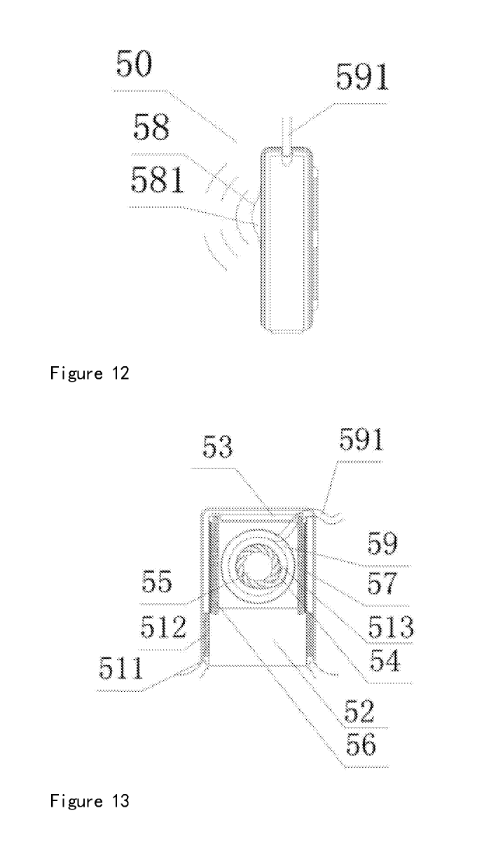

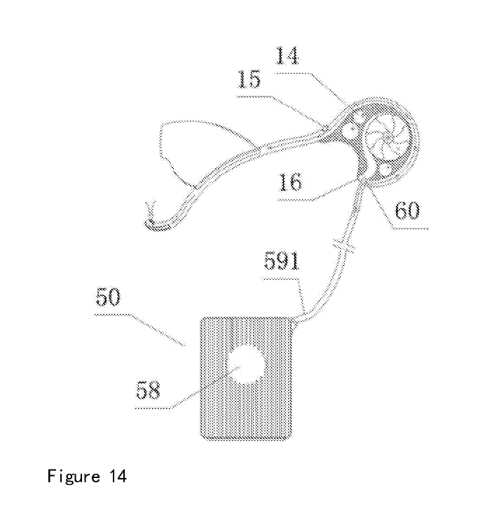

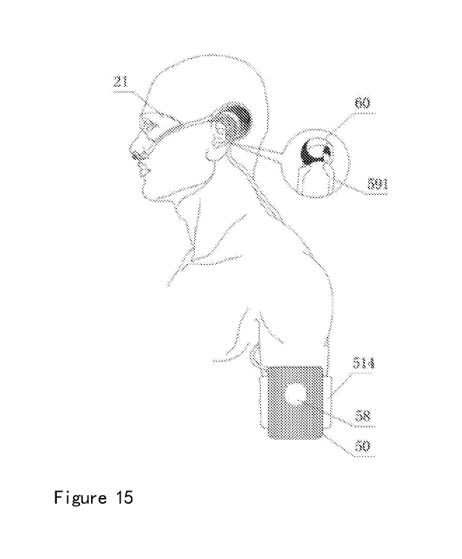

As shown in FIGS. 11-15, based on Embodiments 1-10, the present invention further comprises a portable external air filter 10, the portable external air filter 50 device comprises an intelligent purification integral box 51, a rechargeable battery 52 arranged in the intelligent purification integral box 51, a chip and integral circuit module 53, a centrifugal turbofan vortex housing 54, and a variable-frequency centrifugal turbofan 55, the inner portion of the intelligent clean integral box 51 is provided with a coconut-shell activated carbon filter layer 56, the outer side of the coconut-shell activated carbon filter layer 56 is wrapped with a composite filter layer 57 in a roll-like structure, the side surface of the intelligent purification integral box 51 is provided with a touch control key 58, the touch control key 58 is provided therein with a wireless data induction signal source 581, the lower part of the intelligent purification integral box 51 is provided with a water splashing prevention air inlet channel 511, the water splashing prevention air inlet channel 511 is provided therein with a foam cotton filter layer 512, the intelligent purification integral box 51 is provided therein with a containable stretchable annular coil pipe 59, the intelligent purification integral box 51 is provided therein with an internal pipe connection port 513, the rear side of the intelligent purification integral box 51 is provided with a strap 514, When in use, the strap 514 is bound on an arm, may be fastened by a nylon button or a button, and may also be tied up. The lower portions of the left air filter and the right air filter are provided with the transformation connection port 16, when the present invention uses a short time wearing mode within 1-2 hour working time, the transformation connection port 16 is sealed. the external connection end 591 of the annular coil pipe 59 extends out from the upper portion of the intelligent purification integral box 51, when the present invention requires to use a long time wearing mode within 8-12 hour working time, the external connection end 591 of the annular coil pipe 59 is connected with the transformation connection port 16 provided at the lower portion of the left air filter or the right air filter via a transformation connection head 60 after stretching, At this time, the clean air filtered through the intelligent purification integral box 51 is conveyed to a delivery passage in the left air filter or the right air filter via the annular coil pipe 59, and then conveyed to the air curtain ejection and air transmission device through the left side air transmission pipe 11 or the right side air transmission pipe 12. When not in connection, the annular coil pipe 59 is accommodated in the intelligent purification integral box 51. The portable external air filter 50 uses a more powerful multi-layer air filtration system and an ultra-long time power supply. The portable external air filter 50 is still provided therein with a sharable wireless intelligent interactive data processing chip platform, a wireless transmitter and a wireless signal receiving device are both built in the touch control key 58.

The above-described embodiments are preferred embodiments of the present invention, but the embodiments of the present invention are not limited by the above-described embodiments, any other alterations, modifications, substitutions, combinations, and simplification that do not depart from the spirit and principle of the invention are intended to be equivalents of the present invention and are intended to be included within the scope of the invention.

1. Breathing glasses for purifying air, comprising an ear lug type left air filter and an ear lug type right air filter, a left side air transmission pipe and a right side air transmission pipe which are connected with the left air filter and the right air filter respectively, an air curtain ejection and air transmission device connected with the lower ends of the left side air transmission pipe and the right side air transmission pipe, and an eye protection cover, wherein the left air filter and the right air filter each comprise a filter casing, a composite filter layer disposed within the filter casing, a built-in air inductor, an intelligent induction chip, a dual-channel variable-frequency turbofan, a miniature motor, a drive mechanism and a battery, the filter casing is provided thereon with an air inlet, an air transmission opening and a power control switch;
the eye protection cover comprises an integral transparent material eye protection lens and a bracket provided on the both sides of the integral transparent material eye protection lens, the integral transparent material eye protection lens forms a wearing support frame through the brackets at the two sides thereof that are connected with the left side air transmission pipe and the right side air transmission pipe, respectively;
the air curtain ejection and air transmission device comprises an upper air curtain air outlet groove, a lower air curtain air outlet groove, two breathing air transmission holes and an exhalation pressure or temperature difference induction trigger element, the air exhalation pressure induction trigger element is connected with the intelligent induction chip in the left air filter and the right air filter, the intelligent induction chip detects the respiration rate of a user and air quality, and controls the peak value of the air volume of a variable-frequency nozzle, the exhalation pressure or temperature difference induction trigger element triggers once every time when the user exhales, controls the dual-channel variable-frequency turbofan in the left air filter and the right air filter to delay for a time setting of 0.8-1.2 seconds, then controls the turbofan to rotate at a high speed for a specific time and transfers air to the air curtain ejection and the air transmission device via the left side air transmission pipe and the right side air transmission pipe, the filtered clean air is ejected from the upper air curtain air outlet groove, the lower air curtain air outlet groove and the two breathing air transmission holes of the air curtain ejection and air transmission device, the air ejected from the upper air curtain air outlet groove and the lower air curtain air outlet groove forms an air curtain insulation layer which blocks the surrounding unclean air, a small amount of clean air, which is continuously ejected from the two breathing air transmission holes, causes the nostril portions of the user to form a miniature positive pressure zone to better isolate the surrounding polluted air; a large amount of the clean air, which is continuously ejected from the two breathing air transmission holes synchronously at a suction rhythm by a variable frequency, is sucked by the user.

2. The breathing glasses for purifying the air according to claim 1, wherein the variable frequency turbofan in the left air filter and the right air filter is controlled to delay for one second and then to be rotated at a high speed, and the air is transferred to the air curtain ejection and air transmission device via the left side air transmission pipe and the right side air transmission pipe for 2-3 seconds, which is synchronized with the user's suction rhythm.

3. The breathing glasses for purifying the air according to claim 1, wherein the air curtain ejection device comprises a rigid pipe body, both ends of the rigid pipe body are communicated with the left side air transmission pipe and the right side air transmission pipe, respectively, the outer side of the rigid pipe body is provided with a rigid caulking groove, the cross section of the inner portion of the rigid caulking groove is a circular arc shape, the neck of the rigid caulking groove is reduced, an opening of the outer portion of the rigid caulking groove is enlarged, the value of the angle range of the opening of the outer side of the rigid caulking groove is between 140°-170°, the rear portion of the rigid caulking groove is provided with an air transmission channel communicated with a pipe cavity of the rigid pipe body, a flexible air curtain guide plate made of a flexible material is embedded in the rigid caulking groove, a gap is remained between the outer wall of the flexible air curtain guide plate and the inner wall of the rigid caulking groove to form an air curtain channel, the flexible air curtain guide plate is provided with a multi-point anchor adapted to the rigid caulking groove, the flexible air curtain guide plate is blocked and secured in the rigid caulking groove via the multi-point anchor, the flexible air curtain guide plate and the gap at the opening of the rigid caulking groove form the upper air curtain air outlet groove and the lower air curtain air outlet groove, the two breathing air transmission holes are provided at the upper portion of the rigid pipe body corresponding to nostrils, the exhalation pressure or temperature difference induction trigger element is provided between the two breathing air transmission holes.

4. The breathing glasses for purifying the air according to claim 3, wherein the integral transparent material eye protection lens is provided therein with a visual air monitoring display wafer and an outdoor exercise display wafer, the outer walls of the left side air transmission pipe and the right side air transmission pipe are covered with a C-shaped plastic plate buckle, the integral transparent material eye protection lens is embedded and blocked into a slot between the C-shaped plastic plate buckle and the walls of the left side air transmission pipe and the right side air transmission pipe through the brackets at the two sides thereof respectively to be integrally connected with the left side air transmission pipe and the right side air transmission pipe.

5. The breathing glasses for purifying the air according to claim 4, wherein the outer surface of the flexible air curtain guide plate is provided as an arc surface matched with a nose shape, the outer arc surface of the flexible air curtain guide plate coincides with the missing arc surface at the opening of the rigid caulking groove of the rigid pipe body, forming that the air outlet groove in different parts presents a three-dimensional multi-angle-change multi-curved-surface air curtain layer matched with the nose shape and part of a cheek contour along a notch direction.

6. The breathing glasses for purifying the air according to claim 5, wherein the composite filter layer comprises an outermost mesh coarse filter layer, an intermediate layer ultrafine filter layer and a bottom activated carbon odor filter layer.

7. The breathing glasses for purifying the air according to claim 6, wherein the left side air transmission pipe and the right side air transmission pipe comprise an air transmission pipe bracket made of a hard plastic or a composite material, the dual-channel variable-frequency turbofan is made of the flexible material, the transformation connection ports at the two sides thereof are made of the flexible material.

8. The breathing glasses for purifying the air according to claim 7, wherein the filter casing comprises an elastic silica bottom vortex housing and a metal mussel-shaped gill-like air inlet housing, the air inlet housing in a mussel-shaped gill-like structure is provided with an air inlet trench inclining backwards, a large number of hidden tiny air inlet holes is distributed in the air inlet trench, an inner side surface contacting a head adopts an inclined exhaust trench made of the flexible material to reduce the perspiration of the skin of the contact surface, the dual-channel variable-frequency turbofan is provided with a main channel and a central auxiliary channel, the main channel of the dual-channel variable-frequency turbofan is always kept to be rotated at a low speed and transfer the air without being triggered when in an operating state; the miniature motor is mounted on a cavity-shaped embedded type base made of the flexible material, a small amount of the transferred air in the center auxiliary channel of the dual-channel variable-frequency turbofan is used to cool the miniature motor, the cavity-shaped embedded type base made of the flexible material reduces the vibration and noise of the miniature motor, the intelligent induction chip built in a cavity-shaped space carries out synchronization cooling, the outer portion of the cavity-shaped embedded type base of the flexible material is provided with a heat dissipation vent distributed in the inclined trench.

9. The breathing glasses for purifying the air according to claim 1, wherein the breathing glasses further comprises a portable external air filter, the portable external air filter device comprises an intelligent purification integral box, a rechargeable battery arranged in the intelligent purification integral box, a chip and integral circuit module, a centrifugal turbofan vortex housing, and a two-channel variable-frequency centrifugal turbofan, the inner portion of the intelligent purification integral box is provided with a coconut-shell activated carbon filter layer, the outer side of the coconut-shell activated carbon filter layer is wrapped with a composite filter layer in a roll-like structure, the side surface of the intelligent purification integral box is provided with a touch control key, the touch control key is provided therein with a wireless data induction signal source, the lower part of the intelligent purification integral box is provided with a water splashing prevention air inlet channel, the water splashing prevention air inlet channel is provided therein with a foam cotton filter layer, the intelligent purification integral box is provided therein with an annular coil pipe capable of stretching and contracting, the intelligent purification integral box is provided with an internal pipe connection port, the rear side of the intelligent purification integral box is provided with a strap;
the lower portions of the left air filter and the right air filter are provided with a transformation connection port, the external connection end of the built-in annular coil pipe extends out from the upper portion of the intelligent purification integral box, when in connection, the external connection end of the annular coil pipe is connected with the transformation connection port provided at the lower portion of the left air filter or the right air filter via a transformation connection head after stretching, and converts a working mode of 8-12 hour air transmission of the externally connected intelligent purification integral box; when not in connection, the annular coil pipe is accommodated in the intelligent purification integral box.

10. The breathing glasses for purifying the air according to claim 9, wherein the touch control key of the portable external air filter is provided therein with a wirelessly transmitting device, a wireless signal receiving device and a wireless intelligent interactive data processing chip platform.
